(19) United States
(12) Patent Application Publication
(10) Pub. No.: US 2019/0314550 A1
(43) Pub. Date: Oct. 17, 2019

(54) BONE SUBSTITUTE COMPOSITION AND RELATED METHODS

(71) Applicant: Brahm Holdings, LLC, Germantown, TN (US)

(72) Inventor: Timothy R. BRAHM, Germantown, TN (US)

(21) Appl. No.: 16/385,240

(22) Filed: Apr. 16, 2019

Related U.S. Application Data
(60) Provisional application No. 62/658,169, filed on Apr. 16, 2018.

(51) Int. Cl. A61L 27/36; A61L 27/54

(52) U.S. Cl. CPC A61L 27/3608; A61L 27/365; A61L 2300/602; A61L 2430/02; A61L 2300/252; A61L 27/54

(57) ABSTRACT

A substitute composition is provided that includes at least one cadaveric material and at least one liquid component which, upon introduction to the cadaveric material, causes the cadaveric material to expand and become flowable. Methods for stabilizing a vertebral space are also provided.

### CROSS-REFERENCE TO RELATED APPLICATIONS

The present application claims priority to U.S. Ser. No. 62/658,169 filed Apr. 16, 2018, the contents of which are incorporated in their entirety.

### FIELD OF THE INVENTION

A bone substitute composition is provided. Methods for use of the bone substitute composition are also provided.

### BACKGROUND OF THE INVENTION

The spine has seven cervical, twelve thoracic and five lumbar segments. The bony vertebral bodies of the spine are separated by intervertebral discs. The typical vertebra has a thick anterior bone mass called the vertebral body, with a vertebral arch that arises from the posterior surface of the vertebral body. The spinal disc and vertebral bodies may be displaced or damaged due to trauma, disease, degenerative defects, or wear over an extended period of time. To address back pain, the disc can be removed along with all or part of at least one neighboring vertebrae and is replaced by an implant that promotes fusion of the remaining bones. A fusion element such as a spacer, implant or cage may be used to fill the space left by the removed disc and bony anatomy. A spacer includes an area for the placement of a bone graft to enhance the fusion between the adjacent vertebrae.

There remains a need for safe, effective bone substitute compositions that are compressible, expandable and may serve as vectors for delivery of therapeutic agents.

### SUMMARY OF THE INVENTION

According to one aspect, a bone substitute composition is provided. The bone substitute composition includes at least one cadaveric material selected from the group consisting of cancellous bone, demineralized cancellous bone, fresh allograft, frozen allograft freeze dried bone allograft, demineralized freeze dried bone allograft, cortical cancellous bone, or a combination thereof. The bone substitute composition further includes at least one liquid component which, upon introduction to the cadaveric material, causes the cadaveric material to expand and become flowable. According to one embodiment, the at least one liquid component is selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, donor blood, platelet rich plasma, a flowable birth tissue composition, and a combination thereof. According to one embodiment, the bone substitute composition further includes one or more natural or recombinant bone morphogenetic proteins (BMPs). According to one embodiment, the bone morphogenetic protein is BMP-2, BMP-7 or a combination thereof. According to one embodiment, the bone substitute composition further includes one or more synthetic-based bone graft extenders. According to one embodiment, the bone substitute composition is formulated as a flowable putty to fill a bone void and expandable upon introduction of the at least one liquid component. According to one embodiment, the bone substitute composition is shaped as a wedge, wafer, wafer implant gasket or custom sized and shaped to fill a particular vertebral space. According to one embodiment, the bone substitute composition acts as a vector or delivery system for delivering at least one liquid component to a vertebral space.

According to one aspect, a method of stabilizing a vertebral space is provided. The method includes the steps of providing the bone substitute composition as provided herein, and introducing the bone substitute composition to the vertebral space. According to one embodiment, the method includes the step of hydrating the at least one cadaveric material with the at least one liquid component prior to introducing the bone substitute composition. According to one embodiment, the bone substitute composition is adapted to to expand, fill the vertebral space and deliver the liquid component to the vertebral space. According to one embodiment, the liquid component includes one or more vitamins, nutrients, inflammatory inhibitors, antibiotics, cytokines, minerals, growth factors, hyaluronic acids, cellular attractants, scaffolding reagents, antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue composition, and any combination thereof. According to one embodiment, the vertebral space is present anywhere in a mammalian skeletal system. According to one embodiment, the vertebral space is between an implant and a bone. According to one embodiment, the vertebral space is between or around one or more bones. According to one embodiment, the vertebral space is in the spine, foot, knee, hip or shoulder. According to one embodiment, the bone substitute composition is shaped as a wedge, wafer, wafer implant gasket or custom sized to fill a particular vertebral space. According to one embodiment, the wafer is between 0.1 cm and 10 cm thick.

According to one aspect, a method for delivering at least one liquid component to an area of bone in need of treatment is provided. The method includes the step of introducing at least one liquid component to at least one material selected from the group consisting of cancellous bone, demineralized cancellous bone, fresh allograft, frozen allograft freeze dried bone allograft, demineralized freeze dried bone allograft, cortical cancellous bone, and a combination thereof to form a bone substitute composition; and introducing the bone substitute composition to an area of bone in need treatment. The at least one liquid component is delivered in a time release manner. According to one embodiment, the at least one liquid component includes Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue composition, or a combination thereof. According to one embodiment, the liquid component aids in fusing the spinal region.

According to another aspect, a kit is provided. According to one embodiment, the kit includes a bone substitute composition as provided herein, optionally, at least one screw, rod, or combination thereof for securing the bone substitute composition, and instructions for use thereof.

### DETAILED DESCRIPTION OF THE INVENTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

As used herein, “birth tissue” encompasses one or more of the components of a mammalian placental organ including, but not limited to, the umbilical cord, the umbilical cord blood, the chorionic membrane, the amniotic membrane (intact with no layers removed), the amnion membrane, the Wharton's jelly, the amniotic fluid, and other placental gelatins, cells, and extracellular material.

As used herein, “placental tissue components” encompass one or more of the tissue components of a mammalian placental organ including, but not limited to, the umbilical cord, the umbilical cord blood, the chorionic membrane, the amniotic membrane (intact with no layers removed), the amnion membrane, the Wharton's jelly and other placental gelatins, cells and extracellular material.

As used herein, the term “effective amount” refers to an amount of a particular composition sufficient to elicit the desired therapeutic effects.

As used herein, the term “arthrodesis surgery” refers to a procedures for linking, fusing or otherwise welding bones together. Such a surgery may function to prevent motion between vertebral bodies, repair a fracture or stabilize a spinal deformity.

As used herein, the term “decompression surgery” refers to laminectomy, laminotomy, foraminotomy, laminaplasty, or other spinal surgery where spinal fusion or stabilization interventions may be utilized.

As used herein, the term “laminectomy” refers to the surgical procedure for removing the entire lamina, a portion of the facet joints, and any thickened ligaments overlying the spinal cord and nerves.

As used herein, the term “laminotomy” refers to the surgical procedure for removing a small portion of the lamina and ligaments, usually on a single side.

As used herein, the term “foraminotomy” refers to the surgical procedure for removal of bone around the neural foramen and can be performed with a laminectomy or laminotomy.

As used herein, the term “laminaplasty” refers to the surgical procedure for the expansion of the spinal canal by cutting the laminae on one side and swinging the laminae open.

As used herein, the term “C1” and “first cervical vertebra” refer to the atlas or superior-most vertebra in the spinal column which supports the skull, spinal cord and vertebral arteries.

As used herein, the term “C2” and “second cervical vertebra” refer to the axis vertebra or the epistropheus that is the second-uppermost of the vertebrae making up the backbone and allows the head to rotate from its support atop the C1 vertebra where the skull attaches to the neck.

As used herein, the term “C3-C6” and refer to the third to the sixth vertebra sitting just below the C2 axis vertebra and above the C7 vertebrae that are grouped together

As used herein, the term “C7” and “seventh cervical vertebra” refer to the largest and most inferior vertebra in the neck region. The C7 protrudes posteriorly toward the skin at the back of the neck.

As used herein, the term “time release” refers to the delivery of a liquid component gradually over a period of time.

As used herein, the term “biologic” refers to a type of liquid component that aids in the healing cascade, aids in the spinal fusion process, or a combination thereof.

Provided herein are bone substitute compositions. The bone substitute compositions may be compressible and expandable. According to one embodiment, a bone substitute composition as provided herein stabilizes a skeletal area upon placement. According to one embodiment, a bone substitute composition as provided herein may aid in preventing spinal stenosis recurrence and eliminating pain associated with an unstable spine or degenerative discs in the spine. The bone substitute compositions as provided herein are particularly useful for stabilization of the vertebrae after a spine decompression surgery. The bone substitute compositions as provided herein are particularly useful for placement in or around an implant to fill void or space upon hydrations. The bone substitute composition may be coated with a flowable or liquid component such as, for example, a mammalian birth tissue composition. By providing such a coating, adhesion, nerve damage, pain, and graft migration are reduced or eliminated. Further, the incidence of graft rejection is substantially reduced thereby minimizing the potential need for additional surgery. Also provided herein are methods of stabilizing and treating the spine.

A substitute composition as provided herein may be molded to be of various shapes and sizes depending on the ultimate use of the bone substitute composition. According to one embodiment, the bone substitute composition as provided herein may be freeze dried, placed in any void or space in the skeletal system and then hydrated to expand and fill the void or space. The bone substitute composition as provided herein may be custom cut and shaped depending on the target void or space.

According to one embodiment, the bone substitute composition as provided herein may be formulated as a putty. The putty may be flowable or substantially viscous such that the putty may be molded into spaces for voids in the skeletal system that are otherwise impossible to reach with conventional methods. According to one embodiment, the bone substitute composition includes only cadaveric material from a single donor. According to one embodiment, the putty is introduced via a minimally invasive procedure. According to a particular embodiment, the putty is utilized in spinal applications because during minimally invasive spine techniques, the fusion material cannot get to the posterior side of anterior approach procedures and vice versa. The putty as provided herein is formulated flow and fill the space on the posterior side by expanding once hydrated. According to such an embodiment, the putty is hydrated by a liquid component as provided herein. The liquid component may then be delivered to a target space such as a vertebral space.

According to one embodiment, the bone substitute composition as provided herein is of a shape and size to function as a structural support for the spine, typically in place of a disc or bone that was removed (disc replacement). According to one embodiment, the bone substitute composition as provided herein is of a shape and size to function as an onlay that includes a plurality or mass of bone fragments that grow together to stabilize the spine and bridge any joint. According to one embodiment, the bone substitute composition as provided herein is of a shape and size to provide a scaffold or foundation for new bone growth. According to one embodiment, the bone substitute composition as provided herein is of a shape and size to be placed between an upper and lower vertebral end plate resulting in fusion and stabilization of the point of placement.

According to one embodiment, the bone substitute composition as provided herein is of a shape and size to be used in a foot fusion surgery which may be placed in a void, hydrated (expanded) and then trimmed to shape and size.

According to one embodiment, the bone substitute composition as provided herein is of a shape and size to be utilized in a shoulder implant surgery. In such an embodiment, the bone substitute composition may be implanted between the implant and the bone to fill a void or space. According to one embodiment, the bone substitute composition as provided herein is of a shape and size to include a plurality of clips or chads with are placed in or around a void or space anywhere in the skeletal system such that the bone substitute composition expands to fill the void or space upon hydration.

According to a particular embodiment, the bone substitute composition as provided herein is shaped as a wedge. According to such an embodiment, the wedge may be utilized in a knee or hip replacement surgery and placed in or around an implant (e.g., metal implant). The wedge may be utilized to fill a bone void or space in or around the implant. According to a particular embodiment, the wedge may be used as augmentation wedges to file boney defects during total joint replacement. According to one embodiment, the wedge is custom cut or machined according to a particular boney defect.

According to a particular embodiment, the bone substitute composition as provided herein may be molded to a wafer or disc. The wafer may be of various dimensions and shapes depending on the end use. According to a particular embodiment, the wafer is from about 0.1 cm to about 10 cm thick. According to another embodiment, the wafer is from about 0.5 cm to about 5 cm thick. According to another embodiment, the wafer is from about 0.75 cm to about 7.5 cm thick. According to another embodiment, the wafer is about 1 cm thick. The wafer may be freeze dried and then hydrated with one or more liquid component as provided herein. Upon hydration with a liquid component such as, for example, a biologic, the wafer expands and becomes compressible. The wafer may be placed in any bone void throughout the skeletal system. According to one embodiment, the wafer may be custom cut or machined to an exact size and shape as needed to fill a specific space upon hydration. According to one embodiment, the wafer may be utilized for simple bone fusion procedures with minimal exposure to the bony surfaces in need of fusion.

According to a particular embodiment, the bone substitute composition as provided herein may be molded to a wafer implant gasket. According to one embodiment, the wafer may be machined to exact implant tolerances to fill voids left when implants are attached to bone. According to an exemplary embodiment, the wafer implant gasket may be placed on top of a cut tibia during total knee reconstruction to fill any gaps and to speed healing of recipient bone into implant. The wafer may also aid in delivering a liquid component as provided herein to a target space such as a wound implant interface.

According to an one embodiment, a rod (or plate) is used to aid in prevention of movement of the bone substitute composition upon placement. According to one embodiment, prevention of movement allows the bone substitute composition to attach or fuse. According to such an embodiment, screws are placed above and below any bone, such as vertebrae, fused or stabilized by the graft. The screws and rods can be optionally removed at a later time.

According to one embodiment, the bone substitute compositions as provided herein are prepared by compressing demineralized bone matrix or putty into a desired shape and lyophilizing the shaped cancellous bone matrix into a bone graft. According to one embodiment, the resulting graft is substantially clover-leafed shaped cross-section. According to one embodiment, the resulting graft is wedge shaped from top to bottom.

According to one embodiment, the bone substitute compositions as provided herein are prepared by compressing a demineralized cancellous bone matrix or putty in a compression mold, lyophilizing the demineralized cancellous bone matrix in the mold to form a freeze-dried graft matrix or putty, compressing the matrix contained within the compression mold for a time and under conditions sufficient to form a graft, and removing the freeze-dried graft from the mold.

According to one embodiment, the bone substitute compositions as provided herein are prepared by compressing demineralized cancellous bone matrix or putty, freeze-drying the compressed demineralized cancellous bone matrix to form an implant, and cutting the implant to a particular shape.

The bone substitute compositions as provided herein may be made of one or more materials suitable for implantation into the spine of a mammalian patient such as, for example, a human. Materials may be biocompatible with a mammalian patient and/or may have one or more surface coatings or treatments that enhance biocompatible and also reduce or prevent adhesion, nerve damage, pain, and graft migration. Such bone substitute composition materials may include one or more materials having sufficient load capability and/or strength to maintain the desired spacing between vertebrae and provide the desired stability.

According to one embodiment, the bone substitute compositions as provided herein may optionally include one or more of cancellous bone, demineralized cancellous bone, allograft (fresh or fresh-frozen), freeze dried bone allograft, demineralized freeze dried bone allograft, cortical cancellous bone, or a combination thereof. According to one embodiment, any donors of bone are subject to proper screening. According to one embodiment, the bone substitute compositions as provided herein may optionally include demineralized bone matrix (DBM) that has undergone a process whereby the mineral content is removed. According to one embodiment, demineralized bone matrix functions as a bone graft extender. According to one embodiment, demineralized bone matrix may be mixed with autograft bone or other bone provided herein. According to one embodiment, the autograft includes bone cells, proteins, and calcified matrix. The autograft bone may be harvested from any appropriate portion of a donor's body including, but not limited, a donor's iliac crests, rib or spine. According to one embodiment, demineralized bone matrix may be mixed with allograft bone or other bone provided herein. Any allograft donor bone material may be obtained from a tissue bank. According to one embodiment, the allograft donor bone materials is prepared for use by freezing or freeze-drying to limit rejection.

According to one embodiment, the bone substitute compositions as provided herein may optionally include one or more natural or recombinant bone morphogenetic proteins (BMPs) that are used to stimulate new bone growth. According to a particular embodiment, the bone morphogenetic protein is BMP-2 and BMP-7. According to one embodiment, the bone substitute compositions as provided herein that include one or more bone morphogenetic proteins (BMPs) are suitable for spinal fusion such as an anterior lumbar interbody fusion. According to one embodiment, the bone substitute compositions as provided herein that include one or more bone morphogenetic proteins (BMPs) are suitable for anterior spinal fusions.

According to one embodiment, the bone substitute compositions as provided herein may optionally include one or more synthetic-based bone graft extenders such as, for example, calcium phosphate, tricalcium phosphate, phosphate, calcium sulfate, and bioactive glass. According to one embodiment, the synthetic-based bone graft extender is osteoconductive and biodegradable. The one or more synthetic-based bone graft extenders aid in fusion without a risk for disease transfer. According to one embodiment, the bone substitute compositions as provided herein may optionally include bone marrow aspirate. According to one embodiment, the bone substitute compositions as provided herein may optionally include growth factors, such as platelet derived growth factor and TGF-β to enhance bone healing by promoting mesenchymal stem cell and osteoblast proliferation.

According to a particular embodiment, the bone substitute compositions as provided herein are made from demineralized cancellous bone as set forth in U.S. Pub. No. 20120259425, the content of which is incorporated herein by reference in its entirety. According to one embodiment, the donated bone material is harvested, tested and sterilized by an accredited tissue bank.

According to one embodiment, the bone substitute compositions as provided herein may optionally include one or more bioactive agents. Suitable bioactive agents include, but are not limited to, antimicrobials, antibiotics, antimyobacterial, antifungals, antivirals, antineoplastic agents, antitumor agents, agents affecting the immune response, blood calcium regulators, agents useful in glucose regulation, anticoagulants, antithrombotics, antihyperlipidemic agents, cardiac drugs, thyromimetic and antithyroid drugs, adrenergics, antihypertensive agents, cholnergics, anticholinergics, antispasmodics, antiulcer agents, skeletal and smooth muscle relaxants, prostaglandins, general inhibitors of the allergic response, antihistamines, local anesthetics, analgesics, narcotic antagonists, antitussives, sedative-hypnotic agents, anticonvulsants, antipsychotics, anti-anxiety agents, antidepressant agents, anorexigenics, non-steroidal anti-inflammatory agents, steroidal anti-inflammatory agents, antioxidants, vaso-active agents, bone-active agents, osteogenic factors, antiarthritics, diagnostic agents and progenitor cells, or any combination thereof.

According to one embodiment, the bone substitute compositions as provided herein include one or more synthetic polymers. According to one embodiment, the synthetic polymer is a polycaprolactone, collagen and open porosity polylactic acid polymer. According to one embodiment, the synthetic polymer is a hydrated polymer or hydrogel.

According to one embodiment, the bone substitute compositions as provided herein are compressible and, thus, the bone substitute compositions can be flattened or narrowed under pressure. According to one embodiment, the bone substitute compositions as provided herein are expandable and, thus, the bone substitute compositions can increase in size, volume or otherwise become enlarged. According to one embodiment, the bone substitute compositions as provided herein are compressible and expandable as result of introduction of at least one liquid to one or more of the bone substitute composition components. According to one embodiment, at least one liquid component is introduced to the bone substitute composition components while in powder form thus forming a solid. The at least one liquid component includes, but is not limited to, of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a mammalian birth tissue composition, or a combination thereof.

The compressible and expandable bone substitute compositions as provided herein may be coated, treated, or otherwise include an effective amount of a composition including a mammalian birth tissue. According to one embodiment, the mammalian birth tissue composition includes one or more placental tissue components. The mammalian birth tissue composition may be formulated as a resorbable adhesion barrier allograft that is applied to the outer surface of the graft. According to an alternative embodiment, the mammalian birth tissue composition may be formulated as an injectable formulation or a flowable formulation that is introduced directly onto or into the bone substitute compositions (i.e., mixed). According to either embodiment, placental tissue components and amniotic fluid must first be obtained from a seronegative, healthy mammalian mother. Potential birth tissue donors providing informed consent are pre-screened during an examination of pre-natal medical records and blood test results. A comprehensive medical history and behavior risk assessment is obtained from the donor (or donor's owner) prior to donation incorporating U.S. Public Health Service guidelines. Discussions with the physician(s), veterinarian, and/or the donor mother are conducted to identify circumstances that may lead to the exclusion of the donor or donated tissue. Additionally, a physical exam is performed on the donor to determine whether there is evidence of high risk behavior or infection and to determine the overall general health of the donor.

Infectious disease testing of donor blood specimens is performed for each tissue donor on a specimen collected at the time of donation or within seven days prior to or after donation. Advantageously, the methods that are used to screen for a communicable disease follow the regulations as set forth by the Federal Drug Administration and the American Association of Tissue Banks. Exemplary infectious disease testing includes, but is not limited to, antibodies to the human immunodeficiency virus, type 1 and type 2 (anti-HIV-1 and anti-HIV-2); nucleic acid test (NAT) for HIV-1; hepatitis B surface antigen (HBsAg); total antibodies to hepatitis B core antigen (anti-HBc—total, meaning IgG and IgM); antibodies to the hepatitis C virus (anti-HCV); NAT for HCV; antibodies to human T-lymphotropic virus type I and type II (anti-HTLV-I and anti-HTLV-II); and syphilis (a non-treponemal or treponemal-specific assay may be performed).

Mammalian birth tissue is preferably recovered from a full-term Cesarean delivery of a newborn. Alternatively, birth tissue is recovered from a full-term vaginal delivery of a newborn. The subsequent steps of preparing the birth tissue material are performed in a controlled environment (i.e., certified biological safety cabinet, hood or clean room). Instruments, solutions, and supplies coming into contact with the birth tissue material during processing are sterile. All surfaces coming in contact with the birth tissue material intended for transplant are either sterile or draped using aseptic technique.

Once recovered, one or more of the placental tissue components can be removed via a sterile saline solution rinse, blunt dissection, scalpel, or a combination thereof, if necessary. According to one embodiment, the umbilical cord, chorionic membrane, and other gelatins, fluids, cells and extracellular matrix are removed and discarded, leaving the amniotic membrane for further processing. Preferably, the birth tissue material is subject to preparation no more than four hours after recovery to preserve cell viability.

The retained placental tissue components can be placed in a sterile transport solution after aseptic recovery. The sterile transport solution is used to provide an advantageous medium to the natural function of the placental tissue components prior to processing. For example, calcium-rich water can be used as the sterile transport solution to provide a medium to drive undifferentiated cells to become osteogenic when implanted. Throughout the preparation of the birth tissue material, various methods can be used to drive undifferentiated cells to differentiate into specialized cell types including, but not limited to, transport solutions, soaks, particular temperature ranges, and hyperbaric pressure.

The sterile transport solution may include sodium chloride (NaCl) in a concentration range from typically about 0.1% to typically about 35% by weight. The sterile transport solution can also include one or more of Minimum Essential Medium, Dulbecco's Modified Eagle's Medium, Plasma Lyte-A, human albumin 25% solution, calcium-rich water, alkaline ionized water, or acidic ionized water. After delivery to the processing facility, the weight of the placental tissue components can be determined. Thereafter, the placental tissue components can be transferred aseptically to a sterile dish containing Plasma Lyte-A and stored in a quarantine refrigerator pending further processing. The placental tissue components can be removed from the Plasma Lyte-A and cryopreserved according to methods commonly used in the art. According to one embodiment, the cryopreserved components may then be morselized and formulated into an injectable form and/or a flowable material.

The birth tissue material compositions and bone substitute compositions as described herein can be optionally mixed with or administered in combination with bioactive agents such as inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, vectors for gene delivery and hormones.

The bone substitute compositions as provided herein may be used in a variety of surgeries that require bone grafts such as, for example, decompression, fusion and disc replacement procedures. Specific procedures include anterior spinal decompression and fusion, spinal discectomy alone without fusion, and anterior spinal foramenotomy without complete discectomy. According to one embodiment, the bone substitute compositions as provided herein may be used in arthrodesis surgery for linking, fusing or otherwise welding spinal bones together. Such a surgery may function to prevent motion between vertebral bodies, repair a fracture or stabilize a spinal deformity.

The bone substitute compositions as provided herein may be used in decompression surgery in the spine region. Decompression surgery and subsequent placement of the compressible and expandable bone substitute compositions as provided here are typically performed by an orthopedic surgeon. Surgery is initiated by making a skin incision down the middle of the neck over the appropriate C1-C7 vertebrae. The length of the incision depends on the number of vertebrae that will be subject to the decompression surgery procedure chosen. The neck muscles are then split and moved to either side of each lamina exposing the target vertebral area.

During a disc replacement (e.g., total disc arthroplasty) a disc is completely removed and replaced with a bone substitute composition as provided herein such that the bone substitute composition is placed between vertebral plates. According to one embodiment, the C1 disc is replaced. According to another embodiment, any one or more of the C3-C6 discs is replaced. According to one embodiment, the C7 disc is replaced. Such a disc replacement results in restoration of normal height to the spine to allow proper area and spacing for nerves. Disc replacement also aids in neck stability. Optionally, the surgeon may then retract the dural sac and nerve root to remove any bone spurs or thickened ligaments. The facet joints may then be undercut or trimmed. The spinal fusion to stabilize the spine may then be performed.

According to one embodiment, anterior surgery is performed utilizing one or more of the bone substitute compositions as provided herein. Anterior surgery restores lordosis, stabilizes the spine, and decompresses the nerve roots. According to one embodiment, a plane is opened between the carotid artery and the esophagus leading to the anterior aspect of the spine. According to one embodiment, the vertebral disc is then removed in its entirety along with any osteophytes at the posterior aspect of the vertebral body. The evacuated disc is then replaced with a bone substitute composition as provided herein. Any space in or around the disc may be filled with a bone substitute composition as provided herein such as, for example, a putty formulation as described herein.

According to one embodiment, the bone substitute compositions as provided herein may be used to wick in or up and hold one or more liquid components useful in the healing cascade such that, upon placement, the bone substitute compositions deliver, release or elute such one or more liquid components at the area of placement in the spine. According to one embodiment, the bone substitute composition may expand to fill a given space such as, for example, a bone void upon wicking up (or hydration) the one or more liquid components. According to one embodiment, the bone substitute composition slowly delivers, releases or elutes such one or more liquid components over time. Such liquid components useful in the healing cascade include one or more vitamins (calcium, magnesium, vitamin D3, vitamin K, vitamin C, collagen, glucosamine, chondroitin, vitamin B12, iron, copper, zinc, boron, manganese), nutrients, inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue composition, or any combination thereof.

A method of delivering at least one liquid component to an area in need of treatment is provided. The method includes the step of introducing at least one liquid component to at least one material such as, for example, cancellous bone, demineralized cancellous bone, fresh allograft, frozen allograft freeze dried bone allograft, demineralized freeze dried bone allograft, cortical cancellous bone, or a combination thereof to form a bone substitute composition. The method further includes the step of introducing the bone substitute composition to the spinal region of a patient in need treatment. According to one embodiment, the at least one liquid component is delivered in a time release manner. According to one embodiment, the liquid component is delivered over a period of time from about 1 minutes to about 1 week. According to such an embodiment, the bone substitute composition acts as a vector for delivery of a liquid component. According to one embodiment, the at least one liquid component is a biologic such as, for example, one or more vitamins (calcium, magnesium, vitamin D3, vitamin K, vitamin C, collagen, glucosamine, chondroitin, vitamin B12, iron, copper, zinc, boron, manganese), nutrients, inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue composition, or any combination thereof. According to one embodiment, the liquid component aids in fusing the spinal region. According to one embodiment, the liquid component in the healing cascade.

A kit is also provided that includes one or more bone substitute compositions as provided herein. The kit may include various sizes of compressible and expandable bone substitute compositions depending on the location of where the bone substitute composition may be utilized in the spine. The kit may further include tools or other devices useful in selecting, inserting, positioning, and/or securing one or more bone substitute compositions. Tools and devices may include, for example, one or more pins, screws, rods, plates, wires, cables, straps, surgical rope, sutures, or other devices typically used for positioning and securing the bone substitute compositions. The kit may further include at least one liquid component useful in the healing cascade as provided herein. The kit further includes at least one set of instructions.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

We claim:

1. A bone substitute composition comprising
at least one cadaveric material selected from the group consisting of cancellous bone, demineralized cancellous bone, fresh allograft, frozen allograft freeze dried bone allograft, demineralized freeze dried bone allograft, cortical cancellous bone, or a combination thereof; and
at least one liquid component which, upon introduction to the cadaveric material, causes the cadaveric material to expand and become flowable.

2. The bone substitute composition of claim 1, wherein the at least one liquid component includes one or more vitamins, nutrients, inflammatory inhibitors, antibiotics, cytokines, minerals, growth factors, hyaluronic acids, cellular attractants, scaffolding reagents, antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue composition, and any combination thereof.

3. The bone substitute composition of claim 1, further comprising one or more natural or recombinant bone morphogenetic proteins (BMPs).

4. The bone substitute composition of claim 3, wherein the bone morphogenetic protein is BMP-2, BMP-7 or a combination thereof.

5. The bone substitute composition of claim 1, further comprising one or more synthetic-based bone graft extenders.

6. The bone substitute composition of claim 1, wherein the bone substitute composition is formulated as a flowable putty to fill a bone void and expandable upon introduction of the at least one liquid component.

7. The bone substitute composition of claim 1, wherein the bone substitute composition is shaped as a wedge, wafer, wafer implant gasket or custom sized and shaped to fill a particular vertebral space.

8. A method of stabilizing a vertebral space comprising
providing a bone substitute composition as provided in claim 1; and
introducing the bone substitute composition to the vertebral space, whereby the bone substitute composition expands, fills the vertebral space, and delivers the liquid component to the space.

9. The method of claim 8, wherein the liquid component includes one or more vitamins, nutrients, inflammatory inhibitors, antibiotics, cytokines, minerals, growth factors, hyaluronic acids, cellular attractants, scaffolding reagents, antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue composition, and any combination thereof.

10. The method of claim 8, wherein the vertebral space is present anywhere in a mammalian skeletal system.

11. The method of claim 8, wherein the vertebral space is between an implant and a bone.

12. The method of claim 8, wherein the vertebral space is between or around one or more bones.

13. The method of claim 8, wherein the vertebral space is in the spine, foot, knee, hip or shoulder.

14. The method of claim 8, wherein the bone substitute composition is shaped as a wedge, wafer, wafer implant gasket or custom sized to fill a particular vertebral space.

15. The method of claim 14, wherein the wafer is between 0.1 cm and 10 cm thick.

16. The method of claim 8, further comprising the step of hydrating the at least one cadaveric material with the at least one liquid component prior to introducing the bone substitute composition.

17. A method for delivering at least one liquid component to an area of bone in need of treatment comprising
introducing at least one liquid component to at least one material selected from the group consisting of cancellous bone, demineralized cancellous bone, fresh allograft, frozen allograft freeze dried bone allograft, demineralized freeze dried bone allograft, cortical cancellous bone, and a combination thereof to form a bone substitute composition; and
introducing the bone substitute composition to an area of bone in need treatment,
wherein the at least one liquid component is delivered in a time release manner.

18. The method of claim 17, wherein the at least one liquid component includes one or more vitamins, nutrients, inflammatory inhibitors, antibiotics, cytokines, minerals, growth factors, hyaluronic acids, cellular attractants, scaffolding reagents, antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue composition, and any combination thereof.

19. The method of claim 17, wherein the liquid component aids in fusing the spinal region.

20. A kit for stabilizing or treating an area in need of treatment comprising
a bone substitute composition as provided in claim 1;
optionally, at least one screw, rod, or combination thereof for securing the bone substitute composition to the spine; and
instructions for use thereof.
